(19) United States
(12) Patent Application Publication
(10) Pub. No.: US 2026/0266859 A1
(43) Pub. Date: Sep. 10, 2026

(54) METHOD FOR CONFIGURATION MANAGEMENT OF AT LEAST ONE LABORATORY ANALYZER SYSTEM

(71) Applicant: Roche Diagnostics Operations, Inc., Indianapolis, IN (US)

(72) Inventors: Marian David Roetzer, Munich (DE); Florian Frank Schweinberger, Munich (DE)

(21) Appl. No.: 19/662,278

(22) Filed: Apr. 29, 2026

Related U.S. Application Data
(63) Continuation of application No. PCT/EP2024/080556, filed on Oct. 29, 2024.

(30) Foreign Application Priority Data
Oct. 30, 2023 (EP) 23206688.6

(51) Int. Cl. G01N 35/00; H04L 9/32

(52) U.S. Cl. CPC G01N 35/00871; H04L 9/3236

(57) ABSTRACT

A computer-implemented method for configuration management of at least one laboratory analyzer system (114) comprising at least one analytical unit (116) is proposed. The method comprises the following steps:
a)retrieving at least one item of information on an analytical unit configuration of at least one analytical unit (116) of the laboratory analyzer system (114);
b)applying at least one cryptographic function to the item of information on an analytical unit configuration thereby generating a secured item of information on an analytical unit configuration;
c)providing the secured item of information on an analytical unit configuration to at least one database (122) of the laboratory analyzer system (114) and/or to at least one remote central database (118), wherein the remote central database (118) is at the manufacturer's site and/or a cloud database.

### CROSS-REFERENCE TO RELATED APPLICATIONS

This application is a continuation of International PCT Application No. PCT/EP2024/080556 filed on Oct. 29, 2024, which claims priority to European Patent Application No. 23206688.6 filed on Oct. 30. 2023, the disclosures of which are hereby incorporated by reference in their entirety.

### TECHNICAL FIELD

The invention relates to a computer-implemented method for configuration management of at least one laboratory analyzer system, a computer-implemented method for generating an omnichannel measurement result of at least one laboratory analyzer system, an infrastructure, computer programs, computer-readable storage media and computer program products.

### BACKGROUND ART

In known in vitro diagnostic (IVD) laboratory analyzer systems, configuration management only tracks combinations of multiple analytical units (AUs) and control or core units and on an individual AU level the instrument ID together with major hardware changes, system configuration (consumables and reagents) for the assay. However, a combination of measurement data with a detailed system configuration (history and current list of spare parts and build-in components installed in the system, beyond used chemicals) does not exist. Due to this situation, there may be an technical problem regarding the traceability of system components and therefore arriving at the stage of a better control of the systems in the field is not possible and also the number of configurations installed in the field is unknown (complexity of component interference). Also, there is an additional technical challenge regarding the combination of results and configuration requires that on the data handling level the implementation of a secure technology (out of reach for third-parties) for the data management and storage (both at the manufacturer and the costumer).

U.S. Pat No. 10,521,805 B2 describes determining a qualification status of a device in a system. An occurrence of a trigger event for the device is determined. The trigger event is caused by an occurrence of any of a time-based event, a performance-based event, a usage-based event, and an unscheduled event. A user notification is provided to perform a first action responsive to the occurrence. The first action is an action to perform any of a maintenance activity, a repair activity, and a test for the device. The qualification status of the device is updated in accordance with said first action.

US 2017/220756 A1 describes a mobile unit with a memory which is temporarily connected to a medical technology apparatus is temporarily connected to a central storage device for data transmission. When the mobile unit is connected to the medical technology apparatus, a real configuration of the medical technology apparatus is compared with a local virtual image of the configuration of the medical technology apparatus held in the memory of the mobile unit. Depending on this comparison, the local configuration and/or the real configuration are updated. When the mobile unit is connected to the central storage device, the local configuration is compared with a central virtual image of the configuration of the medical technology apparatus. Depending on this comparison, the central configuration and/or the local configuration are updated.

### PROBLEM TO BE SOLVED

It is therefore an objective of the present invention to provide a computer-implemented method for configuration management of at least one laboratory analyzer system, a computer-implemented method for generating an omnichannel measurement result of at least one laboratory analyzer system, an infrastructure, computer programs, computer-readable storage media and computer program products which avoid the above-described disadvantages of known methods, devices, computer programs and computer program products. In particular, the method and devices shall allow increasing traceability of system components.

### SUMMARY

This problem is addressed by a computer-implemented method for configuration management of at least one laboratory analyzer system, a computer-implemented method for generating an omnichannel measurement result of at least one laboratory analyzer system, an infrastructure, computer programs, computer-readable storage media and computer program products with the features of the independent claims. Advantageous embodiments which might be realized in an isolated fashion or in any arbitrary combinations are listed in the dependent claims as well as throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect, a computer-implemented method for configuration management of at least one laboratory analyzer system comprising at least one analytical unit is disclosed.

The term “computer implemented” as used herein is a broad term and is to be given its ordinary and customary meaning to a person of ordinary skill in the art and is not to be limited to a special or customized meaning. The term specifically may refer, without limitation, to a method involving at least one computer and/or at least one computer network. The computer and/or computer network may comprise at least one processor which is configured for performing at least one of the method steps of the method according to the present invention. Preferably each of the method steps is performed by the computer and/or computer network. The method may be performed completely automatically, such as without user interaction. The term “automatically” as used herein is a broad term and is to be given its ordinary and customary meaning to a person of ordinary skill in the art and is not to be limited to a special or customized meaning. The term specifically may refer, without limitation, to a process which is performed completely by means of at least one computer and/or computer network and/or machine, in particular without manual action and/or interaction with a user.

The term “laboratory analyzer system” as used herein is a broad term and is to be given its ordinary and customary meaning to a person of ordinary skill in the art and is not to be limited to a special or customized meaning. The term specifically may refer, without limitation, to at least one environment comprising at least one analytical unit, e.g. a plurality of laboratory instruments such as at least one analyzer and/or at least one instrument configured for analyzing at least one sample, in particular at least one biological sample. The laboratory analyzer system may be configured for in vitro diagnostic (IVD), e.g. the laboratory analyzer system may be configured for in vitro examination of at least one sample derived from the human body, and/or configured for providing information for diagnostic, monitoring or compatibility purposes. The term “system”, as used herein, may generally refer to an arbitrary set of interacting components being configured for interacting in order to perform at least one common task. Specifically, the components of the laboratory system may interact with each other in order to perform at least one laboratory task. The at least two components may be handled independently or may be coupled or connectable. The laboratory analyzer system specifically may be or may comprise an automated laboratory system configured for automatically or semi-automatically processing a plurality of samples, specifically a large number of samples. As an example, the laboratory analyzer system may be or may comprise an automated laboratory analyzer. The laboratory analyzer system may be used in the field of medical laboratories, such as in clinical laboratories or in forensic laboratories, and/or in the field of chemical laboratories, such as in analytic laboratories. For exemplary embodiments of laboratory analyzer systems which may also be used in the context of the present invention, with the modifications as discussed herein, can be for example the Roche cobas® pro analyzer family or others analytical systems. Other laboratory analyzer systems, however, may also be used.

The laboratory analyzer system comprises at least one analytical unit, e.g. a plurality of analytical units.

The term “unit” as used herein is a broad term and is to be given its ordinary and customary meaning to a person of ordinary skill in the art and is not to be limited to a special or customized meaning. The term specifically may e.g. refer, without limitation, to a module of a modulary system.

The term “analytical unit (AU)” as used herein is a broad term and is to be given its ordinary and customary meaning to a person of ordinary skill in the art and is not to be limited to a special or customized meaning. The term specifically may e.g. refer, without limitation, to a unit of the laboratory analyzer system configured for performing at least one analytical function, e.g. configured for analyzing at least one sample. The analytical unit may be designed, for example, to use a sample or part of the sample and a reagent in order to produce a measurable signal, on the basis of which it is possible to determine whether the analyte is present, and if desired in what concentration. The analytical unit comprises a plurality of modules and/or subunits each comprising at least one hardware element. The analytical unit may use a plurality of consumables, e.g. multiwell plates, vessels, liquids, tips, columns, spare parts and the like. At least some of the modules and/or subunits may be operable to execute one or more processing steps or workflow steps on one or more samples. The processing steps may comprise physically executing processing steps such as centrifugation, aliquotation, sample analysis and the like.

The analytical unit may comprise at least one mass analyzer. The term “mass analyzer” as used herein is a broad term and is to be given its ordinary and customary meaning to a person of ordinary skill in the art and is not to be limited to a special or customized meaning. The term specifically may refer, without limitation, to an arbitrary analytical device which is configured for determining or measuring a mass-to-charge ratio of ions. Measurement results may specifically be presented as a mass spectrum, e.g. a plot of intensity as a function of the mass-to-charge ratio and/or intensity as a function of mass transitions. The readout of the analyzer can also be based on other physical process such as absorption, transmission, scattering, phosphorescence and fluorescence and combinations thereof. In addition, the determination of a functional readout may be combined with other orthogonal methods to increase sensitivity or minimize interferences.

The term “sample” as used herein is a broad term and is to be given its ordinary and customary meaning to a person of ordinary skill in the art and is not to be limited to a special or customized meaning. The term specifically may refer, without limitation, to a material suspected of containing an analyte of interest. The sample can be derived from any biological source, such as a physiological fluid, including, blood, saliva, ocular lens fluid, cerebral spinal fluid, sweat, urine, milk, ascites fluid, mucous, synovial fluid, peritoneal fluid, amniotic fluid, tissue, cells or the like. The sample can be pretreated prior to use, such as preparing plasma from blood, diluting viscous fluids, lysis or the like; methods of treatment can involve filtration, distillation, concentration, inactivation of interfering components, and the addition of reagents. A sample may be used directly as obtained from the source or following a pretreatment to modify the character of the sample, e.g. after being diluted with another solution or after having being mixed with reagents e.g. to carry out one or more diagnostic assays like e.g. clinical chemistry assays, immunoassays, coagulation assays, nucleic acid testing, etc. The term “sample” as used herein is therefore not only used for the original sample but also relates to a sample which has already been processed such as pipetted, diluted, mixed with reagents, enriched, having been purified, having been amplified and the like. As used herein, the term “analyte” may refer to the compound or composition to be detected or measured.

The laboratory analyzer system may comprise further units, e.g. at least one database, at least one control and/or processing unit, and/or further laboratory instruments. The term “laboratory instrument” may cover e.g. pre-analytical instruments, post-analytical instruments, and/or analytical instruments. A pre-analytical instrument can usually be used for the preliminary processing of samples or sample vessels. A post-analytical instrument can usually be used for the post-processing of samples like the archiving of samples. The laboratory analyzer system and/or the analytical unit may comprise at least one laboratory instrument selected from the group consisting of: a pipetting device for pipetting a sample and further devices such as a sorting device for sorting samples or sample vessels, a cap removal device for removing caps or closures on sample vessels, a cap fitting device for fitting caps or closures on sample vessels, an aliquoting device for aliquoting samples, a centrifuging device for centrifuging samples, heating device for heating a sample, a cooling device for cooling a sample, a mixing device for mixing a sample, a separation device for isolating an analyte of the sample, a storing device for storing samples, an archiving device for archiving samples, a sample vessel type determination device for determining a sample vessel type, a sample quality determination device for determining a sample quality.

The term “configuration” as used herein is a broad term and is to be given its ordinary and customary meaning to a person of ordinary skill in the art and is not to be limited to a special or customized meaning. The term specifically may refer, without limitation, to hardware and/or software status.

The term “configuration management” as used herein is a broad term and is to be given its ordinary and customary meaning to a person of ordinary skill in the art and is not to be limited to a special or customized meaning. The term specifically may refer, without limitation, to at least one process for establishing and/or maintaining consistency of performance of the laboratory analyzer system. The configuration management may comprise managing changes throughout the laboratory analyzer system. The configuration management may comprise documenting changes. The configuration management may allow for ensuring traceability of hardware and/or software status. The configuration management may comprise checking repeatedly status of the information. The configuration management may comprise making updated and historical information available within at least one database. The configuration management may comprise providing a documented unbroken chain of hardware and/or software status and/or changes. The configuration management may allow revising capability, improving performance, reliability, or maintainability, extending life, reducing cost, and can allow for correcting defects.

The method comprises the following steps:
a) retrieving at least one item of information on an analytical unit configuration of at least one analytical unit of the laboratory analyzer system;
b) applying at least one cryptographic function to the item of information on an analytical unit configuration thereby generating a secured item of information on an analytical unit configuration;
c) providing the secured item of information on an analytical unit configuration to at least one database of the laboratory analyzer system and/or to at least one remote central database, wherein the remote central database is at the manufacturer's site and/or a cloud database.

The method steps may be performed in the given order or may be performed in a different order. Further, one or more additional method steps may be present which are not listed. Further, one, more than one or even all of the method steps may be performed repeatedly.

The present invention can allow encryption of the content of the data, i.e. encryption of information on an analytical unit configuration of at least one analytical unit of the laboratory analyzer system, by using at least one cryptographic function, e.g. a hash function. Communication techniques using VPN, e.g. as disclosed in US 2017/220756 A1 relate to transmission type and not encryption of the content of the data. In other words, if having the corresponding keys, it is possible to listen to the communication of US 2017/220756 A1 and to decode or understand all the information. In contrast, the encryption of the content of the data as proposed by the present application can ensure that, even after decrypting the communication channel, only a hash is discovered which cannot be decrypted which could not be decoded with finite computing power.

As outlined above, step a) comprises retrieving at least one item of information on an analytical unit configuration of at least one analytical unit of the laboratory analyzer system.

The term “analytical unit configuration” as used herein is a broad term and is to be given its ordinary and customary meaning to a person of ordinary skill in the art and is not to be limited to a special or customized meaning. The term specifically may refer, without limitation, to configuration of hardware and/or software relating to the analytical unit. For example, the item of information on an analytical unit configuration comprises information about at least one element selected from the group consisting of: a software version; a firmware version; a hardware configuration; an assay lot, a consumable lot; module information; unit information; a subunit of the analytical unit; a subunit identifier; a position information; position of at least one article or spare part; a serial number; a lot number; a subunit identifier; an installation date; a unique action identifier; a type of analytical unit; an operator or service technician; a defined maintenance or service action protocol; a timestamp; a location; or a dynamic counter.

The term “module information” as used herein is a broad term and is to be given its ordinary and customary meaning to a person of ordinary skill in the art and is not to be limited to a special or customized meaning. The term specifically may refer, without limitation, to information used to differentiate between modules, e.g. in case more than one analytical unit is present.

The term “unit information” as used herein is a broad term and is to be given its ordinary and customary meaning to a person of ordinary skill in the art and is not to be limited to a special or customized meaning. The term specifically may refer, without limitation, to information used to differentiate between different units of the analytical unit such as at least one unit for liquid chromatography (LC), at least one unit for mass spectrometry (MS). T

The term “information about subunit” as used herein is a broad term and is to be given its ordinary and customary meaning to a person of ordinary skill in the art and is not to be limited to a special or customized meaning. The term specifically may refer, without limitation, to information about an element of a unit of the analytical unit such as to further describe one of the High-performance liquid chromatography (HPLC) subunits of the LC unit.

The information about a subunit identifier (ID) may refer to an ID uniquely describing a subunit.

The information about a type of analytical unit may comprise information about a serial of the analytical unit.

The serial number of an article or spare part may comprise a unique number for each individual article or spare part defined by the manufacturer. An article or spare part may be physical entity required for hardware, and/or software and/or firmware functionality of the analyzer. The article or spare part may have direct impact on the omnichanel measurement result or might be used to control the overall state of the analyzer. It includes for example nozzles, pipettes, incubators, temperature control units, sensors, detectors, pumps, syringes, sealings, tubes, pipes, micro controllers, boards and cables.

The position information may comprise a name of position, which is unique per se, and refers to a coordinate in the three dimensional, physical space. Only a single article or spare part can be assigned to a specific position. Articles and spare parts may be defined by the manufacturer.

The information about a serial number and/or lot number may refer to a unique number for each individual spare part defined by the manufacturer. In case of missing serial number, also the lot number might be suitable for e.g. plastic parts.

The information about the installation date may refer to the date of the installation of the article or spare part on that particular position. This information may get updated when spare part is exchanged and may trigger reset of counters.

The information about a unique action ID may refer to a link used for linking local instrument data and at least one external database (Cube, CIR, and the like). The action ID may be descriptive of performed action on the analytical unit and, optionally, additional metadata. This information may be stored externally. This information may comprise comments, involved persons, time stamps, and the like.

For example, step a) comprises providing at least one item of information about general data such as one or more of a time stamp, a sample unique identifier, a system unique identifier, a system configuration unique identifier, a one-way function, or a location.

For example, step a) comprises providing at least one item of information about a system configuration such as information about at least one analytical unit, e.g. unique identifier, in combination with information about reagents and consumables, e.g. information about reagent such as lot and/or unique identifier, information about consumables such as lot and/or unique identifier.

The item of information on an analytical unit configuration may be retrieved in the form of data. The term “data” as used herein is a broad term and is to be given its ordinary and customary meaning to a person of ordinary skill in the art and is not to be limited to a special or customized meaning. The term specifically may refer, without limitation, to information and/or to machine-readable signals or symbols representative for information. The data specifically may be or may comprise one or both of digital data and analogue data. The data may have multiple data types such as integers, strings and so on. The data may be encrypted data.

The term “retrieve” as used herein is a broad term and is to be given its ordinary and customary meaning to a person of ordinary skill in the art and is not to be limited to a special or customized meaning. The term specifically may refer, without limitation, to the process of obtaining data from a data source. The data source may, however, vary, in accordance with the specific application. The retrieving may take place by at least one of the following: downloading data from a database, such as from at least one data storage device, from a web-or cloud-based data storage device; obtaining data via at least one computer network, such as the Internet; obtaining data via at least one wire-based and/or wireless interface. For example, the retrieving in step a) comprises providing the item of information on an analytical unit configuration by at least one user input via at least one human-machine-interface and/or receiving the item of information on an analytical unit configuration from a database. The retrieving may fully or partially take place automatically, such as by automatic download and/or may fully or partially take manually. Semi-automatic retrieving processes are also possible.

As outlined above, step b) comprises applying at least one cryptographic function to the item of information on an analytical unit configuration thereby generating a secured item of information on an analytical unit configuration.

The term “secured” as used herein is a broad term and is to be given its ordinary and customary meaning to a person of ordinary skill in the art and is not to be limited to a special or customized meaning. The term specifically may refer, without limitation, to being inaccessible, e.g. for one or more of a transmitting entity, a receiving entity, or third parties. The term “secured item of information” as used herein is a broad term and is to be given its ordinary and customary meaning to a person of ordinary skill in the art and is not to be limited to a special or customized meaning. The term specifically may refer, without limitation, to immutable information generated by using encryption technology, e.g. one or more of blockchain technology, certificates-public/private keys, hashcodes, and the like. For example, the secured item of information may be a hash value. The term “cryptographic function” as used herein is a broad term and is to be given its ordinary and customary meaning to a person of ordinary skill in the art and is not to be limited to a special or customized meaning. The term specifically may refer, without limitation, to an algorithm configured for a cryptographic application. For example, in step b), the cryptographic function comprises at least one cryptographic hash function. The method comprises applying at least one cryptographic hash function on the item of information on an analytical unit configuration thereby generating a hash value. A hash function may be an arbitrary function that can be used to map data of arbitrary size to fixed-size values. The hash function may be a Secure Hash Algorithm (SHA), such as SHA-1, SHA-2, SHA-3, SHA-256, or Message-Digest Algorithm (MD) such as MD5, RIPEMD-160, Tiger, HAVAL or Whirlpool. Also non-cryptographic hash functions (e.g. xxHash) and password hash functions can be used. The values returned by a hash function are called hash values. The term “hash value”, also denoted as hash, as used herein is a broad term and is to be given its ordinary and customary meaning to a person of ordinary skill in the art and is not to be limited to a special or customized meaning. The term specifically may refer, without limitation, to a numeric value of a fixed length which uniquely representing data. In case the cryptographic function used for generating the secured item of information is known, decryption may be possible.

The item of information on an analytical unit configuration can have multiple data types such as integers, strings and so on. The information of the analytical unit configuration can be either structured or unstructured. In addition, the information might contain multiple data types such as integers, strings, characters, Boolean, arrays, dates, timestamps. The information data structure can also be nested. In this case the data is flattened before conversion into the data string. Step b) may comprise converting the item of information on an analytical unit configuration into at least one data string, converting the data string to bytes and applying a cryptographic hash function to the bytes thereby generating a hash value. For example, the item of information on an analytical unit configuration may be mutated to pure strings in order to be converted to bytes. Afterwards a cryptgraphic hash function, e.g. SHA256, may be applied to the bytes, leading to unique hashes. The hash can also be transferred to hexadecimal or any other format. Also other functions of the SHA family or the MD5 can be used for hash creation, e.g. as outlined above.

Step c) comprises providing the secured item of information on an analytical unit configuration to at least one database of the laboratory analyzer system and/or to at least one remote central database.

The term “providing” as used herein is a broad term and is to be given its ordinary and customary meaning to a person of ordinary skill in the art and is not to be limited to a special or customized meaning. The term specifically may refer, without limitation, to transferring the secured item of information on an analytical unit configuration, e.g. for storing and/or further usage.

The term “database” as used herein is a broad term and is to be given its ordinary and customary meaning to a person of ordinary skill in the art and is not to be limited to a special or customized meaning. The term specifically may refer, without limitation, to an organized collection of data, generally stored and accessed electronically from a computer or computer system. The database may comprise or may be comprised by a data storage device. The database may comprise at least one data base management system, comprising a software running on a computer or computer system, the software allowing for interaction with one or more of a user, an application or the database itself, such as in order to capture and analyze the data contained in the database. The database management system may further encompass facilities to administer the database. The database, containing the data, may, thus, be comprised by a data base system which, besides the data, comprises one or more associated applications.

The database may be configured for storing the secured item of information on an analytical unit configuration. The database may comprise information about allowed configurations and at least one library. A library may comprise individual cation IDs that are allowed for an individual element of the laboratory analyzer system, e.g. the analytical unit. Moreover, the database may be configured for storing secret keys.

As outlined above, the secured item of information may be provided to a database of the laboratory analyzer system such as an internal database. The internal database may be a logbook.

Additionally or alternatively, the secured item of information on an analytical unit configuration is provided to the remote central database. The term “remote database” as used herein is a broad term and is to be given its ordinary and customary meaning to a person of ordinary skill in the art and is not to be limited to a special or customized meaning. The term specifically may refer, without limitation, to a database external to the laboratory analyzer system. For example, the remote central database is at the manufacturer's site and/or a cloud database. The remote central database may be a remote central database of the manufacturer. The laboratory analyzer system, the service computer and the remote central database form a communication network, wherein the laboratory analyzer system, the service computer and the remote central database are nodes of the communication network. The term “central database”, as used herein is a broad term and is to be given its ordinary and customary meaning to a person of ordinary skill in the art and is not to be limited to a special or customized meaning. The term specifically may refer, without limitation, to a database configured for communication with a plurality of elements of a communication network, e.g. with all participants of the communication network. For example, the remote central database may be configured for communication with the laboratory analyzer system and the service computer.

For example, the secured item of information may be provided in accordance to a standard for an electronic exchange of one or more of medical, administrative and financial data between healthcare information systems. For example, the secured item of information is provided in a z-segment of a Health Level (HL) 7 application layer or a z-segment of a system status message. Typically, HL7 segments comprise MSH (message header), PID (patient information), NK1 (next of kin), PV1 (patient visit). With HL7 messages all kinds of healthcare related information to a variety of disparate systems can be communicated. Sometimes HL7 messages need to contain customized data that cannot be included in any defined segment for its message type. To accommodate for this, the HL7 standard may enable system vendors to create a z-segment with customized fields to transmit this data. At least one of these customized fields may be used for the secured item of information, e.g. as string. Z-segments can be placed anywhere in an HL7 message, however are typically located as the last segment in a message. The hash may also be incorporated in official standards and/or regulatory requirements. HL7 is an international Standard of the ANSI (American National Standards Institute) members and also part of the United Nations regulated ISO standardization. This is the basis for a common communication and helps to reduce misunderstandings. The z-segment is defined for example in HL7-v2 for national or company related segments. A z-segment is a message segment that is defined locally and that is not part of the HL7 standard. Z-segments are part of the reason why the HL7 standard is considered a “flexible” standard. You may find that you need to create new fields to support the requirements of your Z-segment.

The providing of the secured item of information on an analytical unit configuration may be performed via at least one communication interface. The term “communication interface” as used herein is a broad term and is to be given its ordinary and customary meaning to a person of ordinary skill in the art and is not to be limited to a special or customized meaning. The term specifically may refer, without limitation, to an item or element forming a boundary configured for transferring information. The communication interface may be configured for transferring information from a computational device, e.g. a computer, such as to send or output information, e.g. onto another device. Additionally or alternatively, the communication interface may be configured for transferring information onto a computational device, e.g. onto a computer, such as to receive information. The communication interface may specifically provide means for transferring or exchanging information. The communication interface may provide a data transfer connection, e.g. WAN, LAN, Bluetooth, NFC, inductive coupling or the like. As an example, the communication interface may be or may comprise at least one port comprising one or more of a network or internet port, a USB-port and a disk drive. For example, the communication interface may be at least one web interface. For example, the data transfer may be performed via the internet.

Steps a) to c) may be performed by at least one service computer, e.g. as part of service workflow and/or maintenance workflow. The term “service computer” as used herein is a broad term and is to be given its ordinary and customary meaning to a person of ordinary skill in the art and is not to be limited to a special or customized meaning. The term specifically may refer, without limitation, to an arbitrary processing device used for performing a service workflow and/or maintenance workflow. The service computer may be a PC, tablet computer and the like.

The term “service workflow and/or maintenance workflow” as used herein is a broad term and is to be given its ordinary and customary meaning to a person of ordinary skill in the art and is not to be limited to a special or customized meaning. The term specifically may refer, without limitation, to any activity intended to retain or restore a functional unit in or to a specified state in which the unit can perform its required function and/or intended purpose. For example, the service workflow and/or maintenance workflow may comprise at least one action selected from the group consisting of: at least one test, at least one measurement, at least one replacement, at least one adjustment, and at least one repair. For example, the service workflow and/or maintenance workflow may comprise one or more of installation of at least one spare part, updates, repairs, maintenance. The service and/or maintenance action can contain physical exchange and/or repairs of spare parts or sub units, completely/partially updating or changing the software(s) and/or firmware(s) of the analytical unit. Also predictive and preventive maintenance actions based on pre-defined procedures can alter the analytical unit.

For example, the service workflow and/or maintenance workflow comprises
performing at least one service and/or maintenance action on the at least one analytical unit;
generating an updated item of information on an analytical unit configuration;
applying the cryptographic function to the updated item of information on an analytical unit configuration thereby generating an updated secured item of information on an analytical unit configuration;
providing the updated secured item of information on an analytical unit configuration to the database of the laboratory analyzer system and to the remote central database.

The method further may comprises providing the secured item of information on an analytical unit configuration to a Laboratory Information Management System (LIMS). The LIMS may be configured for electronically recording measurement results generated by the at least one analytical unit of the laboratory analyzer system. The secured item of information on an analytical unit configuration may be transferred from the service computer to the LIMS and/or may be transferred from a control unit of the laboratory system to the LIMS. The LIMS may transfer the secured item of information to the remote central database.

The method may comprise using static hashes, e.g. a hash which is generated at the service computer and provided to a database unchanged. The method, in addition or alternatively, may use dynamic hashes. For example, additional information may be added to the hash generated at the service computer and may be encrypted afterwards, e.g. by applying the cryptographic function or a further cryptographic function, thereby generating a hash value. The hash value may be send to the database. In case the cryptographic function used for generating the dynamic hash is known, decryption may be possible.

The method may comprise performing steps a) to c) upon installation of the laboratory system thereby establishing an initial secured item of information on an analytical unit configuration;

The method steps may be repeated. For example, the method is repeated upon a subsequent change and/or at a further predefined time thereby generating an updated secured item of information on an analytical unit configuration. Step c) may comprises replacing the secured item of information on an analytical unit configuration by the updated item of information on an analytical unit configuration in the database of the laboratory analyzer system and providing the updated item of information on an analytical unit configuration to the remote central database.

For example, the method may comprise creating a set of dynamic counters to trigger different maintenance actions such as different analytes and/or general usage of one spare part to influence the timing for maintenance.

For example, the method may comprise generating of at least one root-hash. The root-hash may be a top hash, also denoted as master hash, of a hash tree. Generating of the root-hash may comprise repeating steps a) to b) using a further item of information on an analytical unit configuration, generating a further hash value and aggregating the hash value and the further hash value. The method further may comprise generating of a blockchain. The method may comprise generating a plurality of subsequent linked blocks of the blockchain. The term “block”, as used herein is a broad term and is to be given its ordinary and customary meaning to a person of ordinary skill in the art and is not to be limited to a special or customized meaning. The term specifically may refer, without limitation, to a component or an element of the blockchain. Blockchains can be defined as a chain of blocks which can hold certain records and the blocks are linked to one another using the principles of cryptography, for further details see e.g. en.wikipedia.org/wiki/Blockchain. The blockchain may comprise a plurality of linked blocks. The blocks may be linked by comprising a hash value of the previous block. Each block may comprise a cryptographic hash of the previous block. Each block may further comprise a timestamp. Each block may comprise a hash of the previous block, a timestamp and, optionally further data.

The method may comprise an integrity test. The integrity test may comprise retrieving an actual secured item of information on an analytical unit configuration stored in the database of the laboratory analyzer system and an actual secured item of information on an analytical unit configuration stored in the remote central database. The integrity test may comprise comparing the actual secured item of information on an analytical unit configuration stored in the database of the laboratory analyzer system and the actual secured item of information on an analytical unit configuration stored in the remote central database. The integrity test is failed in case the actual secured item of information on an analytical unit configuration stored in the database of the laboratory analyzer system and the actual secured item of information on an analytical unit configuration stored in the remote central database differ, otherwise the integrity test is passed.

The present invention may allow creating a secured item of information such as hash values, i.e. configuration strings, which allow to easily identify past, current and future high level analytical unit configuration status. This can allow covering two pathways at once: historical documentation of each and every change and a tracking of branches that highlights the major changes. As will be outlined in more detail below, the secured item of information can get assigned to each measurement result.

In a further aspect, a computer-implemented method for generating an omnichannel measurement result of at least one laboratory analyzer system is disclosed. The method comprises the following steps
I) Providing at Least One Measurement Result Generated by at Least One Analytical Unit of the laboratory analyzer system;
ii) linking, by using at least one control unit of the laboratory analyzer system, the measurement result with a secured item of information on an analytical unit configuration generated by performing a method for configuration management according to the present invention, such as according to any one of the embodiments disclosed with respect to the method above and/or according to any one of the embodiments disclosed in further detail below, thereby generating the omnichannel measurement result.

With respect to embodiments and definitions reference is made to definitions and embodiments given with respect to the method for configuration management according to the present invention, such as according to any one of the embodiments disclosed with respect to the method above and/or according to any one of the embodiments disclosed in further detail below.

The term “providing at least one measurement result”, as used herein is a broad term and is to be given its ordinary and customary meaning to a person of ordinary skill in the art and is not to be limited to a special or customized meaning. The term specifically may refer, without limitation, to performing at least one measurement with the analytical unit and/or retrieving at least one measurement result from a database, e.g. the database of the analytical unit.

The method may comprise linking the measurement result with additional information obtained by at least one further internal and/or at least one further external sensor. The method may comprise linking the measurement result with at least one additional ambient readout. The additional ambient readout may comprise one or more of current, voltage, temperature, pressure, humidity, light conditions and the like. This readout may be provided by sensors which are part of the analytical unit, e.g. and may be present on the analytical unit in terms of log files, and/or may also comprise data of sensors in closed proximity of the analytical unit. This readout may be used to weighting usage of components, e.g. different climate conditions. This readout may be used to confirm potential out of specification usage, e.g. impact on pipetting performance by different temperatures. For example, the additional information may be retrieved from the remote central database. For example, the additional information may comprise information about one or more of constituents, miscellaneous sensors, overall equipment efficiency, and information from a fault indicating system.

The method may comprise linking the measurement result with additional information obtained by advanced analytics such as using internal sensor storage and/or advanced sensor interpretation.

The term “control unit”, as used herein is a broad term and is to be given its ordinary and customary meaning to a person of ordinary skill in the art and is not to be limited to a special or customized meaning. The term specifically may refer, without limitation, to an arbitrary unit configured for performing the named operations, preferably by using at least one data processing device and, more preferably, by using at least one processor and/or at least one application-specific integrated circuit. Thus, as an example, the at least one control unit may comprise at least one data processing device having a software code stored thereon comprising a number of computer commands. The control unit may provide one or more hardware elements for performing one or more of the named operations and/or may provide one or more processors with software running thereon for performing one or more of the named operations. The control unit may comprise one or more programmable devices such as one or more computers, application-specific integrated circuits (ASICs), Digital Signal Processors (DSPs), or Field Programmable Gate Arrays (FPGAs) which are configured to perform a control function. The control unit may comprise at least one computer. The computer can be an embedded computer e.g. micro controller or programmable logic devices such as FPGAs. Additionally or alternatively, however, the control unit may also fully or partially be embodied by hardware.

The term “linking”, as used herein is a broad term and is to be given its ordinary and customary meaning to a person of ordinary skill in the art and is not to be limited to a special or customized meaning. The term specifically may refer, without limitation, to one or more of combining, connecting, fusing, attaching, aggregating the measurement result and the secured item of information. For example the configuration information may be stored on a single memory device whereas the measurement result is received, processed and stored on the detector unit. The core unit might request both separate information, combine them and send them to an external device.

The term “channel”, as used herein is a broad term and is to be given its ordinary and customary meaning to a person of ordinary skill in the art and is not to be limited to a special or customized meaning. The term specifically may refer, without limitation, to a source of information. The analytical unit may provide a channel referring to as a measurement channel. The item of information on an analytical unit configuration may be provided by at least one channel different from the measurement channel. The item of information on an analytical unit configuration may be provided by at least one channel designed for performing a method for configuration management according to the present invention. The channel different from the measurement channel may comprise at least one data source selected from the group consisting of at least one database such as the remote central database or the database of the laboratory analyzer system, a log file, at least one input by a service client or a service technician via a service computer, and the like. The term “omnichannel measurement result”, as used herein is a broad term and is to be given its ordinary and customary meaning to a person of ordinary skill in the art and is not to be limited to a special or customized meaning. The term specifically may refer, without limitation, to a measurement result provided by the measurement channel comprising additional information from at least one further channel.

The method may comprises one or more of transferring the omnichannel measurement result to at least one further device, logging and/or storing and/or postprocessing the omnichannel measurement result in at least one database, performing at least one maintenance action depending on the omnichannel measurement result, and further analyzing of the omnichannel measurement result such as for weighting usage of components and/or validating the measurement result and/or confirming potential out of specification usage.

The present invention may allow for creating a link in each and every measurement result with the analytical unit configuration. For example, the measurement result may comprise at least information about used materials such as assay lot, consumable lots, software versions, and the like, and/or detailed diagnostic analyzer hardware configuration such as instrument ID, operator or service technician, defined maintenance/service action protocols, positions, spare part UIDs, timestamp and location. The linking of the measurement result to the additional information mentioned above can occur in a secure way by ensuring secure data transfer as well as immutable and permanent storage using encryption technology such as blockchain, certificates—public/private keys, hashcodes, etc. This can allow ensuring improved serviceability of laboratory analyzer systems by using configuration management and advanced empirical data supported analytics for spare parts.

The present invention may have the following advantages: Full traceability and log of all information that contributes to a patient result can be assured. It is possible to comply with regulatory requirements (medical device). The omnichannel measurement result can ensure a higher standard for patient result safety. A faster serviceability in the field (systems status is known) can be assured and improved preventive maintenance actions can be defined. It may be possible to only do necessary maintenance to the extend what is needed, i.e. dynamic maintenance, e.g. split and/or define maintenance actions based on individual usage and/or conditions such as combination of different counters and/or condition of components used in this action, e.g. variation of acid concentration to clean to the necessary amount and not beyond to extend system life. Predictive maintenance may be possible to auto order spare parts with expected end of life until next service visit therefore prolonging instrument uptime. It may be possible to protect internal instrument know-how and logs from third party access while simultaneously making information available on-side.

In a further aspect, an infrastructure is disclosed. The infrastructure comprises
at least one service computer configured for performing the method for configuration management according to the present invention, such as according to any one of the embodiments disclosed with respect to the method above and/or according to any one of the embodiments disclosed in further detail below;
at least one remote central database;
at least one laboratory analyzer system comprising at least one analytical unit, wherein the analytical unit comprises at least one analytical instrument, wherein the laboratory analyzer system comprises at least one control unit and at least one database, wherein the laboratory analyzer system comprises at least one communication interface configured for communicating with the remote central database;
wherein the infrastructure is configured for performing the method for generating an omnichannel measurement result according to the present invention, such as according to any one of the embodiments disclosed with respect to the method above and/or according to any one of the embodiments disclosed in further detail below, using the analytical unit and the control unit of the laboratory analyzer system.

With respect to embodiments and definitions reference is made to definitions and embodiments given with respect to the methods according to the present invention, such as according to any one of the embodiments disclosed with respect to the methods above and/or according to any one of the embodiments disclosed in further detail below.

Further disclosed and proposed herein is a computer program including computer-executable instructions for performing the method for configuration management and/or the method for generating an omnichannel measurement result according to the present invention in one or more of the embodiments enclosed herein when the instructions are executed on a computer or computer network. Specifically, the computer program may be stored on a computer-readable data carrier and/or on a computer-readable storage medium.

As used herein, the terms “computer-readable data carrier” and “computer-readable storage medium” specifically may refer to non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

Thus, specifically, one, more than one or even all of method steps as indicated above may be performed by using a computer or a computer network, preferably by using a computer program.

Further disclosed and proposed herein is a computer program product having program code means, in order to perform the method for configuration management and/or the method for generating an omnichannel measurement result according to the present invention in one or more of the embodiments enclosed herein when the program is executed on a computer or computer network. Specifically, the program code means may be stored on a computer-readable data carrier and/or on a computer-readable storage medium.

Further disclosed and proposed herein is a data carrier having a data structure stored thereon, which, after loading into a computer or computer network, such as into a working memory or main memory of the computer or computer network, may execute the method for configuration management and/or the method for generating an omnichannel measurement result according to one or more of the embodiments disclosed herein.

Further disclosed and proposed herein is a non-transient computer-readable medium including instructions that, when executed by one or more processors, cause the one or more processors to perform the method for configuration management and/or the method for generating an omnichannel measurement result.

Further disclosed and proposed herein is a computer program product with program code means stored on a machine-readable carrier, in order to perform the method for configuration management and/or the method for generating an omnichannel measurement result according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network.

Finally, disclosed and proposed herein is a modulated data signal which contains instructions readable by a computer system or computer network, for performing the method for configuration management and/or the method for generating an omnichannel measurement result according to one or more of the embodiments disclosed herein.

Referring to the computer-implemented aspects of the invention, one or more of the method steps or even all of the method steps of the method for configuration management and/or the method for generating an omnichannel measurement result according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, further disclosed herein are:
a computer or computer network comprising at least one processor, wherein the processor is adapted to perform one or both of the methods according to one of the embodiments described in this description,
a computer loadable data structure that is adapted to perform one or both of the methods according to one of the embodiments described in this description while the data structure is being executed on a computer,
a computer program, wherein the computer program is adapted to perform one or both of the methods according to one of the embodiments described in this description while the program is being executed on a computer,
a computer program comprising program means for performing one or both of the methods according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform one or both of the methods according to one of the embodiments described in this description after having been loaded into a main and/or working storage of a computer or of a computer network, and
a computer program product having program code means, wherein the program code means can be stored or are stored on a storage medium, for performing one or both of the methods according to one of the embodiments described in this description, if the program code means are executed on a computer or on a computer network.

Summarizing and without excluding further possible embodiments, the following embodiments may be envisaged:

Embodiment 1. A computer-implemented method for configuration management of at least one laboratory analyzer system comprising at least one analytical unit, wherein the method comprises the following steps:
a) retrieving at least one item of information on an analytical unit configuration of at least one analytical unit of the laboratory analyzer system;
b) applying at least one cryptographic function to the item of information on an analytical unit configuration thereby generating a secured item of information on an analytical unit configuration;
c) providing the secured item of information on an analytical unit configuration to at least one database of the laboratory analyzer system and/or to at least one remote central database, wherein the remote central database is at the manufacturer's site and/or a cloud database.

Embodiment 2. The method according to the preceding embodiment, wherein the item of information on an analytical unit configuration comprises information about at least one element selected from the group consisting of: a software version; a firmware version; a hardware configuration; an assay lot, a consumable lot; module information; unit information; a subunit of the analytical unit; a subunit identifier; a position information; position of at least one article or spare part; a serial number of an article or spare part; a lot number; an installation date; a unique action identifier; a type of analytical unit; an operator or service technician; a defined maintenance or service action protocol; a timestamp; a location; or a dynamic counter.

Embodiment 3. The method according to any one of the preceding embodiments, wherein the retrieving in step a) comprises providing the item of information on an analytical unit configuration by at least one user input via at least one human-machine-interface and/or receiving the item of information on an analytical unit configuration from a database.

Embodiment 4. The method according to any one of the preceding embodiments, wherein the method comprises using dynamic hashes, wherein additional information is added to the hash generated in step b) and is encrypted afterwards thereby generating a hash value.

Embodiment 5. The method according to any one of the preceding embodiments, wherein, in step b), the cryptographic function comprises at least one cryptographic hash function, wherein the method comprises applying at least one cryptographic hash function on the item of information on an analytical unit configuration thereby generating a hash value.

Embodiment 6. The method according to the preceding embodiment, wherein step b) comprises converting the item of information on an analytical unit configuration into at least one data string, converting the data string to bytes and applying a cryptographic hash function to the bytes thereby generating a hash value.

Embodiment 7. The method according to any one of the two preceding embodiments, wherein the method further comprises generating of at least one root-hash, wherein generating of the root-hash comprises repeating steps a) to b) using a further item of information on an analytical unit configuration, generating a further hash value and aggregating the hash value and the further hash value.

Embodiment 8. The method according to any one of the three preceding embodiments, wherein the method further comprises generating of a blockchain, wherein the method comprises generating a plurality of subsequent linked blocks of the blockchain, wherein each block comprises a hash of the previous block and a timestamp.

Embodiment 9. The method according to any one of the preceding embodiments, wherein the method further comprises providing the secured item of information on an analytical unit configuration to a Laboratory Information Management System (LIMS), wherein the LIMS is configured for electronically recording measurement results generated by the at least one analytical unit of the laboratory analyzer system.

Embodiment 10. The method according to any one of the preceding embodiments, wherein the secured item of information is provided in a z-segment of an HL7 application layer or a z-segment of a system status message.

Embodiment 11. The method according to any one of the preceding embodiments, wherein the method comprises performing steps a) to c) upon installation of the laboratory system thereby establishing an initial secured item of information on an analytical unit configuration.

Embodiment 12. The method according to any one of the preceding embodiments, wherein the method comprises at least one service workflow and/or maintenance workflow, wherein the method comprises
performing at least one service and/or maintenance action on the at least one analytical unit;
generating an updated item of information on an analytical unit configuration;
applying the cryptographic function to the updated item of information on an analytical unit configuration thereby generating an updated secured item of information on an analytical unit configuration;
providing the updated secured item of information on an analytical unit configuration to the database of the laboratory analyzer system and to the remote central database.

Embodiment 13. The method according to any one of the preceding embodiments, wherein the method comprises an integrity test, wherein the integrity test comprises retrieving an actual secured item of information on an analytical unit configuration stored in the database of the laboratory analyzer system and an actual secured item of information on an analytical unit configuration stored in the remote central database, wherein the integrity test comprises comparing the actual secured item of information on an analytical unit configuration stored in the database of the laboratory analyzer system and the actual secured item of information on an analytical unit configuration stored in the remote central database, wherein the integrity test is failed in case the actual secured item of information on an analytical unit configuration stored in the database of the laboratory analyzer system and the actual secured item of information on an analytical unit configuration stored in the remote central database differ, otherwise the integrity test is passed.

Embodiment 14. The method according to any one of the preceding embodiments, wherein the method is repeated upon a subsequent change and/or at a further predefined time thereby generating an updated secured item of information on an analytical unit configuration, wherein step c) comprises replacing the secured item of information on an analytical unit configuration by the updated item of information on an analytical unit configuration in the database of the laboratory analyzer system and providing the updated item of information on an analytical unit configuration to the remote central database.

Embodiment 15. The method according to any one of the preceding embodiments, wherein step a) comprises retrieving at least one item of information about general data such as one or more of a time stamp, a sample unique identifier, a system unique identifier, a system configuration unique identifier, a one-way function, or a location.

Embodiment 16. The method according to any one of the preceding embodiments, wherein step a) comprises retrieving at least one item of information about a system configuration such as information about at least one analytical unit, e.g. unique identifier, in combination with information about reagents and consumables, e.g. information about reagent such as lot and/or unique identifier, information about consumables such as lot and/or unique identifier.

Embodiment 17. A computer-implemented method for generating an omnichannel measurement result of at least one laboratory analyzer system, wherein the method comprises the following steps
i) providing at least one measurement result generated by at least one analytical unit of the laboratory analyzer system;
ii) linking, by using at least one control unit of the laboratory analyzer system, the measurement result with a secured item of information on an analytical unit configuration generated by performing a method for configuration management according to any one of the preceding embodiments, thereby generating the omnichannel measurement result.

Embodiment 18. The method according to the preceding embodiment, wherein the method comprises linking the measurement result with at least one additional ambient readout, wherein the additional ambient readout comprises one or more of current, voltage, temperature, pressure, humidity, light conditions and the like.

Embodiment 19. The method according to any one of the preceding embodiments, wherein the method comprises one or more of transferring the omnichannel measurement result to at least one further device, logging and/or storing and/or postprocessing the omnichannel measurement result in at least one database, performing at least one maintenance action depending on the omnichannel measurement result, and further analyzing of the omnichannel measurement result such as for weighting usage of components and/or validating the measurement result and/or confirming potential out of specification usage.

Embodiment 20. An infrastructure comprising
at least one service computer configured for performing the method according to any one of embodiments 1 to 16;
at least one remote central database;
at least one laboratory analyzer system comprising at least one analytical unit, wherein the analytical unit comprises at least one analytical instrument, wherein the laboratory analyzer system comprises at least one control unit and at least one database, wherein the laboratory analyzer system comprises at least one communication interface configured for communicating with the remote central database;
wherein the infrastructure is configured for performing the method according to any one of embodiments 17 to 19 using the analytical unit and the control unit of the laboratory analyzer system.

Embodiment 21. A computer program comprising instructions which, when the program is executed by the processing unit such as of a service computer, cause the processing unit to perform the method according to any one of embodiments 1 to 16.

Embodiment 22. A computer-readable storage medium comprising instructions which, when the instructions are executed by the processing unit such as of a service computer, cause the processing unit to perform the method according to any one of embodiments 1 to 16.

Embodiment 23. A non-transient computer-readable medium including instructions that, when executed by one or more processors, cause the one or more processors to perform the method according to any one of embodiments 1 to 16.

Embodiment 24. A computer program comprising instructions which, when the program is executed by the infrastructure according to any one of the preceding embodiments referring to a infrastructure, cause the infrastructure to perform the method according to any one of embodiments 17 to 19.

Embodiment 25. A computer-readable storage medium comprising instructions which, when the instructions are executed by the infrastructure according to any one of the preceding embodiments referring to an infrastructure, cause the infrastructure to perform the method according to any one of embodiments 17 to 19.

Embodiment 26. A non-transient computer-readable medium including instructions that, when executed by one or more processors, cause the one or more processors to perform the method according to any one of embodiments 17 to 19.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIG. 1 shows an infrastructure overview;

FIG. 2 shows a flowchart of an exemplary embodiment of a method for configuration management;

FIG. 3 shows a flowchart of a workflow overview of a method for configuration management;

FIG. 4 shows an embodiment of step a) of the method of retrieving an item of information on an analytical unit configuration;

FIG. 5 shows an embodiment of a method for generating an omnichannel measurement result; and

FIG. 6 shows an embodiment of an analytical unit configuration.

### DETAILED DESCRIPTION OF THE EMBODIMENTS

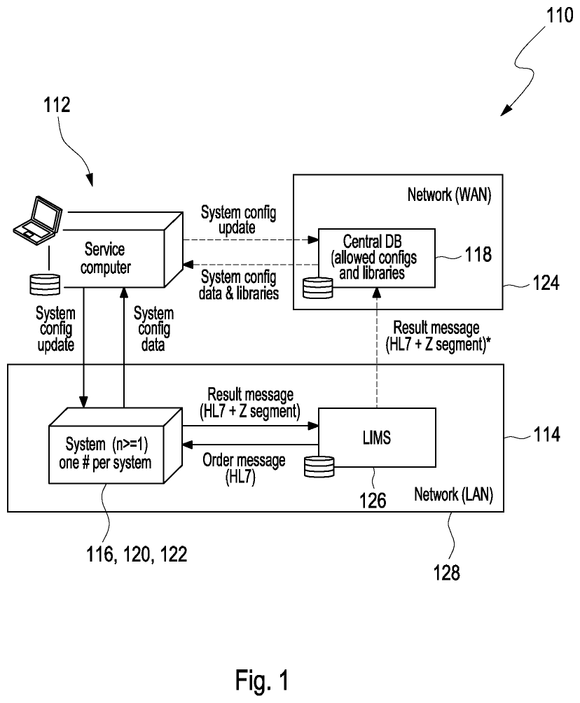

FIG. 1 shows an infrastructure 110 overview. The infrastructure 110 comprises at least one service computer 112 configured for performing a computer-implemented method for configuration management of at least one laboratory analyzer system 114.

The laboratory analyzer system 114 may be configured for in vitro diagnostic (IVD), e.g. the laboratory analyzer system 114 may be configured for in vitro examination of at least one sample derived from the human body, and/or configured for providing information for diagnostic, monitoring or compatibility purposes. The laboratory analyzer system 114 may be or may comprise an automated laboratory system configured for automatically or semi-automatically processing a plurality of samples, specifically a large number of samples. As an example, the laboratory analyzer system 114 may be or may comprise an automated laboratory analyzer. The laboratory analyzer system 114 specifically may be used in the field of medical laboratories, such as in clinical laboratories or in forensic laboratories, and/or in the field of chemical laboratories, such as in analytic laboratories. The laboratory analyzer system 114 comprises at least one analytical unit (AU) 116, e.g. a plurality (n) of analytical units 116 (n≥1). For example, the analytical unit 116 may comprise at least one mass analyzer. The sample can be derived from any biological source, such as a physiological fluid, including, blood, saliva, ocular lens fluid, cerebral spinal fluid, sweat, urine, milk, ascites fluid, mucous, synovial fluid, peritoneal fluid, amniotic fluid, tissue, cells or the like. The sample can be pretreated prior to use, such as preparing plasma from blood, diluting viscous fluids, lysis or the like; methods of treatment can involve filtration, distillation, concentration, inactivation of interfering components, and the addition of reagents. The laboratory analyzer system 114 may comprise further units further laboratory instruments, e.g. pre-analytical instruments, post-analytical instruments, and/or analytical instruments.

The infrastructure 110 further comprises at least one remote central database 118. The remote central database 118 may be external to the laboratory analyzer system 114. For example, the remote central database 118 is at the manufacturer's site and/or a cloud database. The laboratory analyzer system 114, the service computer 112 and the remote central database 118 form a communication network 124, wherein the laboratory analyzer system 114, the service computer 112 and the remote central database 118 are nodes of the communication network 124. The remote central database 118 may be configured for communication with all participants of the communication network 124. For example, the remote central database 118 may be configured for communication with the laboratory analyzer system 114 and the service computer 112, e.g. via WAN communication.

As show in FIG. 1, the laboratory analyzer system 114 comprises at least one control unit 120 and at least one database 122. The laboratory analyzer system 114 may comprise a Laboratory Information Management System (LIMS) 126. The LIMS 126 may be configured for electronically recording measurement results generated by the at least one analytical unit 116. The laboratory analyzer system 114 may comprise an internal communication network 128 configured for communication between elements of the laboratory analyzer system 114, such as between the analytical unit 116, the control unit 120 and the database 122, e.g. via LAN. The laboratory analyzer system 114 comprises at least one communication interface configured for communicating with the remote central database 118. For example, as shown in FIG. 1 the laboratory analyzer system 114 may communicated via the LIMS 126 with the remote central database 118.

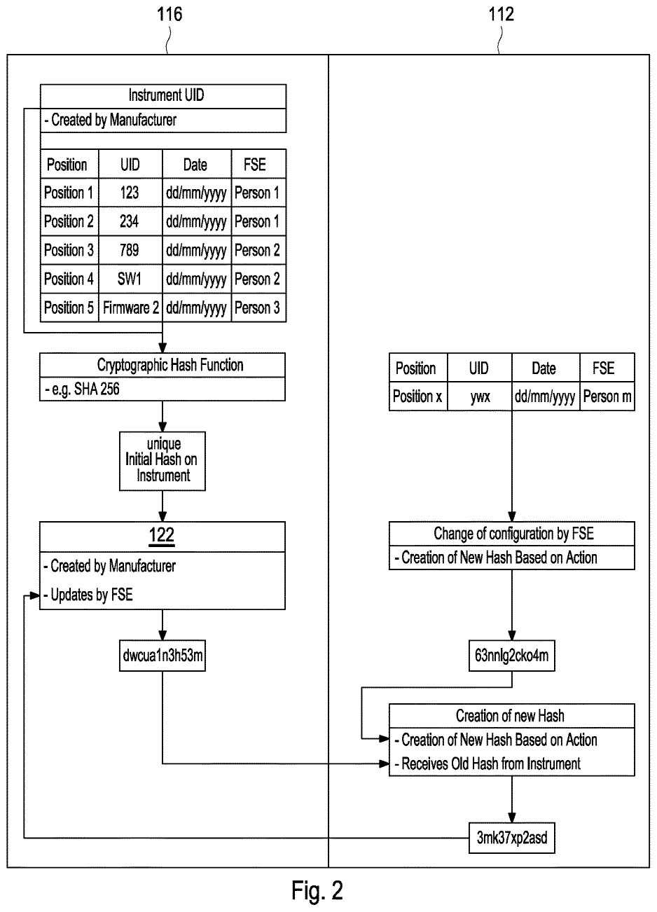

FIG. 2 shows a flowchart of an exemplary embodiment of a method for configuration management as performed by the infrastructure 110, as shown in FIG. 1. The method for configuration management makes used of at least one cryptographic functions to log configuration and history of the analytical unit 116. In FIG. 2, exemplarily, the method steps are distinguished into steps performed by the analytical unit 116 and the service computer 112. A workflow overview from a perspective of service technician or Field Systems Engineer (FSE) perspective is shown.

In a first step, e.g. at installation of the instrument, an instrument unique identifier may be defined by the manufacturer. In addition, initial analytical unit configuration such as position, unique identifier (UID), time stamp and information about the FSE may be generated. For example, as shown in FIG. 2, the initial analytical unit configuration comprises information about three (spare) parts at positions 1 to 3 with UID “123”, “234” and “789”, at position 4 software (SW1) and at position 5 firmware (Firmware2) and information about three FSEs involved. A start hash (“unique initial hash on the instrument”) may be established by applying a cryptographic hash function, e.g. SHA 256, to the item of information on an analytical unit configuration. For example, the start hash in FIG. 2 is “dwcua1n3h53m”. The start hash may be stored in the database 122.

For example as part of service workflow and/or maintenance workflow, the FSE “m” may change an element of the analytical unit 116, e.g. at position x, at certain time. The analytical unit configuration is changed, e.g. in the logbook, and a hash value is generated based on this action, e.g. “63nnlg2cko4m”. An updated hash is created using this hash value and the last available hash from the database 122, e.g. “3mk37xp2asd”. The change on the system is stored on the analytical unit 116, e.g. within the logbook. Thus, each change gets a unique hash based at minimum on timestamp, instrument ID, action description and last available hash. Additionally, the hash gets stored at the remote central database 118. Any changes on the analytical unit 116 will corrupt the hash and be revealed by a non-sense logbook.

For example as shown in FIG. 1, the hash may be provided in accordance to a standard for an electronic exchange of one or more of medical, administrative and financial data between healthcare information systems. For example, the hash is provided in a z-segment of a Health Level (HL) 7 application layer or a z-segment of a system status message. Typically, HL7 segments comprise MSH (message header), PID (patient information), NK1 (next of kin), PV1 (patient visit). With HL7 messages all kinds of healthcare related information to a variety of disparate systems can be communicated. Sometimes HL7 messages need to contain customized data that cannot be included in any defined segment for its message type. To accommodate for this, the HL7 standard may enable system vendors to create a z-segment with customized fields to transmit this data. At least one of these customized fields may be used for the hash, e.g. as string. Z-segments can be placed anywhere in an HL7 message, however are typically located as the last segment in a message.

As shown in FIG. 1, the service computer 112 may generate a system configuration update and communicate the system configuration update to the analytical unit 116. The service computer 112 may receive the analytical unit configuration, denoted as “System config data” in FIG. 1. The service computer 112 may receive from the remote central database 118 stored system configuration data and libraries. The service computer 112 may send a system configuration update to the remote central database 118. The analytical unit 116 may send the HL7 message (result message comprising the HL7 and the Z-segment) in response to an order by the LIMS 126. The LIMS 126 may provide the result message to the remote central database 118.

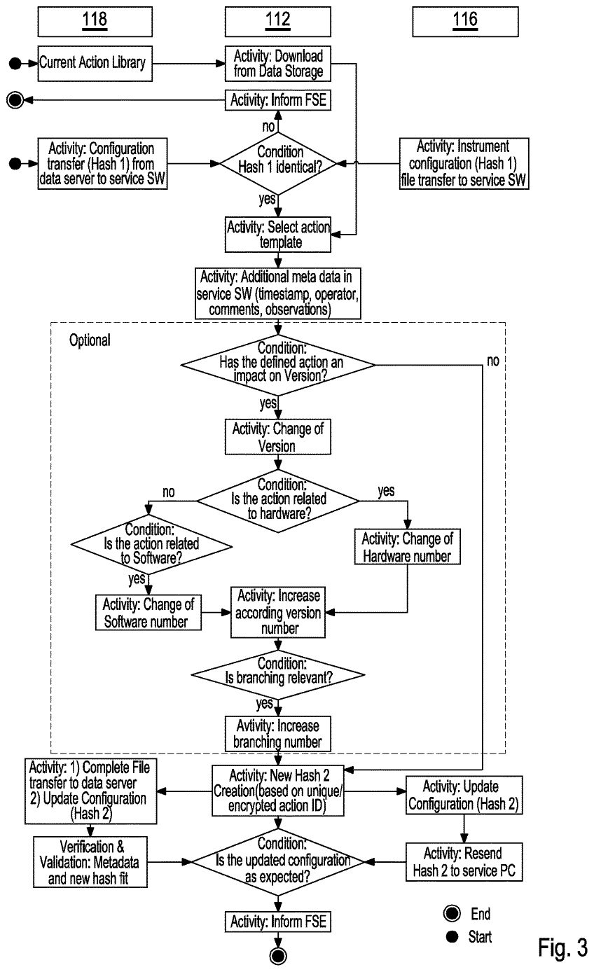

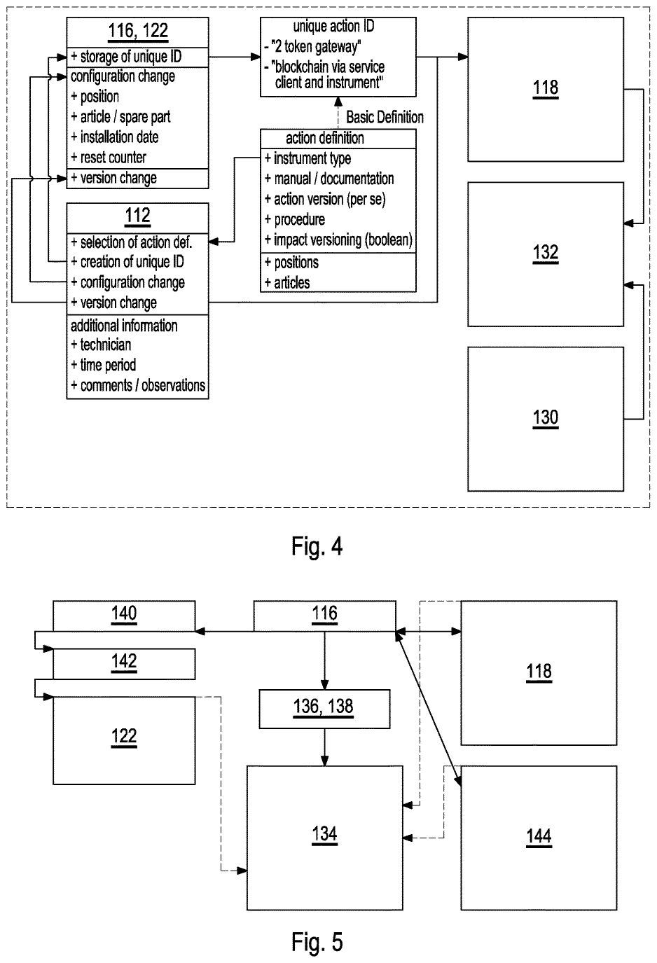

FIG. 3 shows a flowchart of a workflow overview for service/maintenance by FSE or an operator. For example, the FSE visits a customer in order to do maintenance. Firstly, the workflow may comprise step a) of the method for configuration management, retrieving at least one item of information on an analytical unit configuration of at least one analytical unit 116 of the laboratory analyzer system 114. The service computer 112 may receive from the remote central database 118 a current action library, comprising potential actions on the analytical unit 116. In addition, a latest hash (Hash 1) may be transferred from the remote central database 118 to the service computer 112 and a latest hash is transferred from the analytical unit 116 to the service computer 112. The method may comprises an integrity test in which the Hash 1 from the remote central database 118 and the latest hash from the analytical unit 116 are compared. In case the two hashes are not identical the FSE may be informed. In case the two hashes are identical, for example, via a user interface, e.g. a GUI and service software, the FSE can select an action, which is performed on the analytical unit 116. For example, the action to be performed on the analytical unit 116 is selected as an action template. For example, the action on the analytical unit 116 may comprise replacing of an old spare part and installation of a new spare part. The action on the analytical unit 116 may comprise updating the cumulative log with a new spare part, installation date and serial numbers. A unique action identifier (abbreviated as action ID) which may be an entry of the item of information on an analytical unit configuration, may be created externally by the FSE. An embodiment of creating an action ID is shown in FIG. 4. The FSE may input additional information into the service computer 112 such as additional meta data, e.g. time stamp, operator comments, and observations. The old spare part may be replaced locally and the fate of the spare part may be saved and/or updated externally. The installation of the new spare part may lead to reset of counters.

Optionally, it is checked by the service computer 112 if the defined action has an impact on the version (hardware or software). If no, the workflow may continue with creating a hash value, as will be described below. If yes, the version may be changed. The service computer 112 may check if the action is related to hardware or software version, and may change depending on the check the hardware number or the software number (or both) and increases according to the version number. In addition, the service computer 112 may check if branching is relevant and may increase a branching number.

Next, the workflow may comprise step b) of the method for configuration management comprising applying at least one cryptographic function to the item of information on an analytical unit configuration, in this embodiment the action ID, thereby generating a secured item of information on an analytical unit configuration. In particular, a hash value (Hash 2) is created, e.g. based on the unique action ID and/or an encrypted action ID.

Next, the workflow may comprise step c) of the method for configuration management comprising providing the secured item of information on an analytical unit configuration to the database 122 and/or to the remote central database 118. As shown in FIG. 3, three options to proceed may exist.

For example, as shown with the arrow to the left, the next activity may comprise 1) completing a file transfer to the remote central database 118, and 2) updating the analytical unit configuration in the form of the Hash 2. Next, a verification and validation may be performed using metadata and comprising checking if the hash value fits, e.g. is as expected. If the updated configuration is as expected the FSE is informed.

For example, as shown with the arrow in the middle, the next activity may comprise checking if the updated configuration is as expected only. If the updated configuration is as expected the FSE is informed.

For example, as shown with the arrow to the right, the next activity may comprise updating the analytical unit configuration in the form of the Hash 2 within the database 122. Next, the Hash 2 may be resend to the service computer 112 and it is checked if the updated configuration is as expected only. If the updated configuration is as expected the FSE is informed.

FIG. 4 shows an embodiment of creating an action ID.

Potential actions performable by a service client may be defined by basic definitions. The basic definitions may comprise information about one or more of instrument type, manual or documentation, action version, procedure, impact on versioning (Boolean), positions and articles. The basic definitions may be provided to the FSE in the form of action templates from which the service client can select.

The service client via the service computer 116 can select one of the action definitions. In addition, the service client may create a unique ID, perform a configuration change, and optionally a version change. Moreover, the service client via the service computer 116 can input additional information such as about one or more of the technician, a time period, comments and observations.

The analytical unit 116 may store, e.g. in database 122, the unique ID (as generated by the service client). The analytical unit 116 may undergo a configuration change (as performed by the service client). The configuration change may be defined by on ore more of position, article or spare part, and installation date. The configuration change may comprise resetting counter(s). The configuration change may comprise a version change (as indicated by the service client).

The action ID may be created by a 2 token gateway and/or a blockchain such as via a service client and the instrument.

The action ID may be transferred to the remote central database 118 for central storage. The additional information from the database 122 or the service computer 112 may be transferred to the remote central database 118 for central storage.

The remote central database 118 may provide information, e.g. about the analytical unit configuration, to further entities such as to a warehouse server 132 of a warehouse 130. The warehouse 130 may be a physical and/or digital warehouse, e.g. of a combined instrument and article and/or spare part warehouse. The warehouse 130 may be configured for providing information relating to one or more of storage place, delivery dates, labels to the warehouse sever 132. The information may be stored in a spare part logbook.

FIG. 5 shows an embodiment of a method for generating an omnichannel measurement result 134 of the laboratory analyzer system 114. The method comprises step i) 136 comprising providing at least one measurement result 138 generated by the analytical unit 116, e.g. performing at least one measurement. The analytical unit 116 may provide a channel referring to as measurement channel. The analytical unit 116 in FIG. 5 may comprise at least one module 140 having at least one subunit 142. The database 122 may comprise information about the configuration such as one or more of position, spare part, date, counter and the item of information on an analytical unit configuration (e.g. the action ID).

The method further comprises step ii) comprising linking, by using at least one control unit 120, the measurement result 138 with a secured item of information on an analytical unit configuration generated by performing a method for configuration management according to the present invention, e.g. as described with respect to FIGS. 1 to 4, thereby generating the omnichannel measurement result 136. The linking may comprise one or more of combining, connecting, fusing, attaching, aggregating the measurement result 138 and the secured item of information.

The item of information on an analytical unit configuration may be provided by at least one channel different from the measurement channel. For example, the item of information on an analytical unit configuration may be retrieved from the database 122.

The method may comprise linking the measurement result 138 with additional information obtained by at least one further internal and/or at least one further external sensor. For example, method may comprise linking the measurement result with at least one additional ambient readout. The additional ambient readout comprises one or more of current, voltage, temperature, pressure, humidity, light conditions and the like. For example, the additional information may be retrieved from the remote central database 118. For example, the additional information may comprise information about one or more of constituents, miscellaneous sensors, overall equipment efficiency, and information from a fault indicating system.

The method may comprise linking the measurement result 138 with additional information obtained by advanced analytics 144 such as using internal sensor storage and/or advanced sensor interpretation.

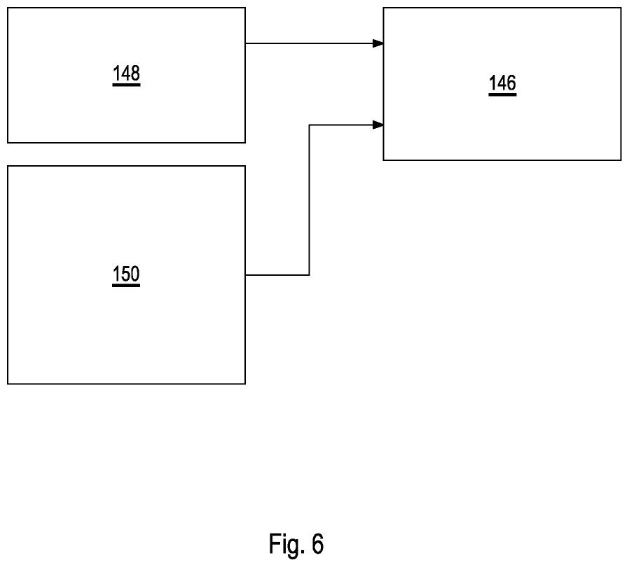

FIG. 6 shows an embodiment of an analytical unit configuration 146. The analytical unit configuration 146 may comprise information about the position 148. The position may relate to a defined set of potential positions. The position may define a unique location with the analytical unit 116. Some positions may even be restricted for certain articles or spare parts. Each position is only available for a single article or spare part. No double occupation is allowed.

The analytical unit configuration 146 may comprise information about the article or spare part 150, such as one or more of an article number, available positions, available spare parts, serial number or lot number or additional information.

Thus, the resulting analytical unit configuration 146 may comprise information the position 148, information about the article or spare part 150, an action ID, time stamp and counter. A counter can either be classical events such as switching events, injections or the like, or calculated events such as amount of organic solvent or an accumulated acid content.

### LIST OF REFERENCE NUMBERS

110 infrastructure
112 service computer
114 laboratory analyzer system
116 analytical unit
118 remote central database
120 control unit
122 database
124 communication network
126 LIMS
128 internal communication network
130 warehouse
132 warehouse sever
134 omnichannel measurement result
136 step i)
138 measurement result
140 module
142 subunit
144 advanced analytics
146 analytical unit configuration
148 position
150 information about the article or spare part

1. A computer-implemented method for configuration management of at least one laboratory analyzer system comprising at least one analytical unit, the method comprising:
retrieving at least one item of information on an analytical unit configuration of at least one analytical unit of the laboratory analyzer system;
applying at least one cryptographic function to the item of information on the analytical unit configuration thereby generating a secured item of information on the analytical unit configuration, wherein applying the at least one cryptographic function comprises applying at least one cryptographic hash function on the item of information on the analytical unit configuration thereby generating a hash value, wherein additional information is added to the generated hash value and is encrypted by applying the at least one cryptographic function or a further cryptographic function afterwards thereby generating a dynamic hash value;
providing the secured item of information on the analytical unit configuration to at least one database of the laboratory analyzer system and/or to at least one remote central database, wherein the remote central database is at a manufacturer's site and/or a cloud database.

2. The method according to claim 1, wherein the item of information on an analytical unit configuration comprises information about at least one element selected from the group consisting of: a software version; a firmware version; a hardware configuration; an assay lot, a consumable lot; module information; unit information; a subunit of the analytical unit; a subunit identifier; a position information; position of at least one article or spare part; a serial number of an article or spare part; a lot number; an installation date; a unique action identifier; a type of analytical unit; an operator or service technician; a defined maintenance or service action protocol; a timestamp; a location; or a dynamic counter.

3. The method according to claim 1, further comprising generating a blockchain, including generating a plurality of subsequent linked blocks of the blockchain, wherein each block comprises a hash of the previous block and a timestamp.

4. The method according to claim 1, wherein the secured item of information is provided in a z-segment of an HL7 application layer or a z-segment of a system status message.

5. The method according to claim 1, wherein the method comprises at least one service workflow and/or maintenance workflow, the method further comprising:
performing at least one service and/or maintenance action on the at least one analytical unit;
generating an updated item of information on the analytical unit configuration;
applying the cryptographic function to the updated item of information on the analytical unit configuration thereby generating an updated secured item of information on the analytical unit configuration; and
providing the updated secured item of information on the analytical unit configuration to the database of the laboratory analyzer system and to the remote central database.

6. The method according to claim 1, further comprising performing an integrity test that comprises retrieving an actual secured item of information on the analytical unit configuration stored in the database of the laboratory analyzer system and an actual secured item of information on the analytical unit configuration stored in the remote central database and comparing the actual secured item of information on the analytical unit configuration stored in the database of the laboratory analyzer system and the actual secured item of information on the analytical unit configuration stored in the remote central database, wherein the integrity test is failed in case the actual secured item of information on the analytical unit configuration stored in the database of the laboratory analyzer system and the actual secured item of information on the analytical unit configuration stored in the remote central database differ, otherwise the integrity test is passed.

7. The method according to claim 1, wherein the method of claim 1 is repeated upon a subsequent change and/or at a further predefined time thereby generating an updated secured item of information on the analytical unit configuration, wherein providing the secured item of information on the analytical unit configuration comprises replacing the secured item of information on the analytical unit configuration by the updated item of information on the analytical unit configuration in the database of the laboratory analyzer system and providing the updated item of information on the analytical unit configuration to the remote central database.

8. A computer-implemented method for generating an omnichannel measurement result of at least one laboratory analyzer system, the method comprising:
providing at least one measurement result generated by at least one analytical unit of the laboratory analyzer system;
linking, by using at least one control unit of the laboratory analyzer system, the measurement result with a secured item of information on an analytical unit configuration generated by performing a method for configuration management according to claim 1, thereby generating the omnichannel measurement result.

9. An infrastructure, comprising:
at least one laboratory analyzer system comprising at least one analytical unit, wherein the at least one analytical unit comprises at least one analytical instrument, at least one control unit, at least one database, and at least one communication interface configured for communicating with a remote central database; and
at least one computing system configured to (i) retrieve at least one item of information on an analytical unit configuration of the at least one analytical unit, (ii) apply at least one cryptographic function to the item of information on the analytical unit configuration to generate a secured item of information on the analytical unit configuration, wherein to apply the at least one cryptographic function comprises to apply at least one cryptographic hash function on the item of information on the analytical unit configuration thereby generating a hash value, wherein additional information is added to the generated hash value and is encrypted by applying the at least one cryptographic function or a further cryptographic function afterwards thereby generating a dynamic hash value, and (iii) provide the secured item of information on the analytical unit configuration to the at least one database and/or to the remote central database, wherein the remote central database is at a manufacturer's site and/or a cloud database.

10. The infrastructure of claim 13, wherein the at least one laboratory analyzer system is configured to generate an omnichannel measurement result by (i) providing at least one measurement result generated by the at least one analytical unit and (ii) linking, by using the at least one control unit, the measurement result with a secured item of information on the analytical unit configuration, thereby generating the omnichannel measurement result.

11. At least one non-transitory machine-readable storage medium comprising a plurality of instructions stored thereon that, in response to execution by at least one processing unit, causes a computing system to:
retrieve at least one item of information on an analytical unit configuration of at least one analytical unit;
apply at least one cryptographic function to the item of information on the analytical unit configuration to generate a secured item of information on the analytical unit configuration, wherein to apply the at least one cryptographic function comprises to apply at least one cryptographic hash function on the item of information on the analytical unit configuration thereby generating a hash value, wherein additional information is added to the generated hash value and is encrypted by applying the at least one cryptographic function or a further cryptographic function afterwards thereby generating a dynamic hash value; and
provide the secured item of information on the analytical unit configuration to at least one database and/or to remote central database, wherein the remote central database is at a manufacturer's site and/or a cloud database.
